(19) United States
(12) Patent Application Publication
(10) Pub. No.: US 2014/0296838 A1
(43) Pub. Date: Oct. 2, 2014

(54) RADIO FREQUENCY HEATING DEVICE

(71) Applicant: METAL INDUSTRIES RESEARCH&DEVELOPMENT CENTRE, KAOHSIUNG (TW)

(72) Inventors: YI-SAN CHANG, Tainan City (TW); SHIH-HUA HUANG, Kaohsiung City (TW); YU-SUNG HSIEH, Tainan City (TW); SHYANG-JYE CHANG, New Taipei City (TW); SHEAU-CHIOU CHAO, Tainan City (TW)

(73) Assignee: METAL INDUSTRIES RESEARCH&DEVELOPMENT CENTRE, KAOHSIUNG (TW)

(21) Appl. No.: 14/092,107

(22) Filed: Nov. 27, 2013

(30) Foreign Application Priority Data
Mar. 29, 2013 (TW) 102111436

(51) Int. Cl. A61B 18/08

(52) U.S. Cl. CPC A61B 18/082
USPC 606/30

(57) ABSTRACT

A radio frequency (RF) heating device includes a flexible pad, a plurality of electrodes and an RF generator unit. The electrodes are disposed on one side of the flexible pad for emitting an RF energy. The RF generator unit is electrically connected to the electrodes for providing the electrodes with the RF energy.

### CROSS-REFERENCE TO RELATED APPLICATION

This application claims the benefit of Taiwan Patent Application No. 102111436, filed on Mar. 29, 2013, which is hereby incorporated by reference for all purposes as if fully set forth herein.

### BACKGROUND OF THE INVENTION

1. Field of Invention

The present invention relates to a radio frequency (RF) heating device, and more particular to an RF heating device in which electrodes are disposed on one side of a flexible pad.

2. Related Art

Currently, for most people, skin problems are treated with a therapy treatment apparatus by means of energy transmission, such as a laser, a radio frequency, an infrared ray, an LED, an aerodynamic force, etc. for the treatments of pigmentation removal, hair removal, wrinkle removal, rejuvenation, and anti-aging in order to receive effects of medical cosmetics.

The RF heating technology has been widely used. In early stage, it mainly uses in a variety of medical surgeries. However, the special effects caused by RF heating such as skin tissue contract for slimming beauty have recently been found, so that the RF heating can be applied to the field of medical cosmetics. Nowadays, most of RF heating apparatuses have a column-like therapeutic device in common, and the therapeutic device may be provided with ten or less electrodes therein. When the therapeutic device contacts human skin, it can guide RF energy into the skin tissues. Besides, in order to make skin absorb the heat energy, doctors usually move the therapeutic device back and forth on the treating skin area to heat up the skin tissues. However, during the treatment, it may take a long time to move the therapeutic device back and forth on the treating skin area in order to make more skin absorb the heat, such that doctors may easily surfer muscle fatigue and stiffness, which may affect the effect and quality of treatment. Also, the medical expense may increase as the treatment time takes longer for patients. Moreover, if the treatment is for a large skin area, the treating time and expenses may increase tremendously.

Therefore, an RF heating device used for a large skin area is needed in order to overcome the above-mentioned problems.

### SUMMARY OF THE INVENTION

One objective of the present invention is to provide an RF heating device for a large skin area and face skin area.

To achieve the objective, the present invention provides a radio frequency (RF) heating device including a flexible pad, a plurality of electrodes and an RF generator unit. The electrodes are disposed on one side of the flexible pad for emitting an RF energy. The RF generator unit is electrically connected to the electrodes for providing the electrodes with the RF energy.

The RF heating device of the present invention can be used in an automatic medical cosmetics treatment for large skin area, and is different from conventional RF heating products. The RF heating device of the present invention can guide RF energy to skin tissues by using a plurality of electrodes disposed on a flexible pad, so as to raise the temperatures of skin tissues to promote the contracts of dermal collagen, elastic fibers and other tissues due to heat absorption. In addition, the area of the flexible pad is adjustable and can be cut into a shape corresponding to each part of human body. For example, the flexible pad can be cut into a shape similar to face. Moreover, when the RF heating device of the present invention is used in a treatment for a large skin area, for example, abdominal region, it can reduce the treating time.

The present invention will become more fully understood from the detailed description given herein below for illustration only, and thus are not limitative of the present invention.

### BRIEF DESCRIPTION OF THE DRAWINGS

FIG. 1 is a schematic perspective view of an RF heating device according to a first embodiment of the present invention.

FIG. 2 is a structural block diagram of an RF heating device according to a first embodiment of the present invention.

FIG. 3 is a top view showing electrodes arrangement according to one embodiment of the present invention.

FIG. 4 is a side view of electrodes and a flexible pad of FIG. 3.

FIG. 5 is a cross-section view of an enlarged portion A of FIG. 4.

FIGS. 6 and 7 are schematic perspective views of an RF heating device according to a second embodiment of the present invention respectively showing a front side and a back side of a mask-type flexible pad.

FIG. 8 is a schematic view showing an RF heating device in use according to the first and the second embodiment of the present invention.

### DETAILED DESCRIPTION OF THE INVENTION

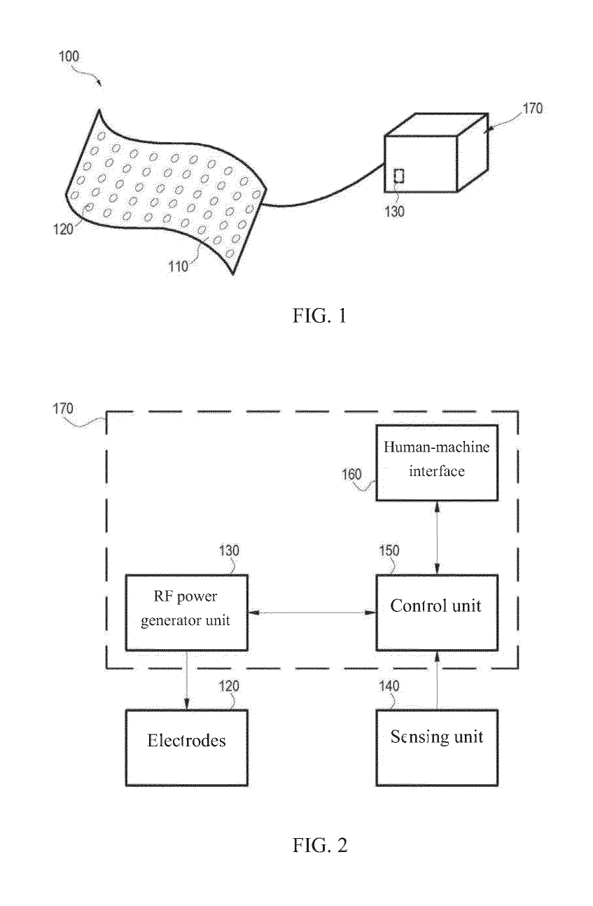

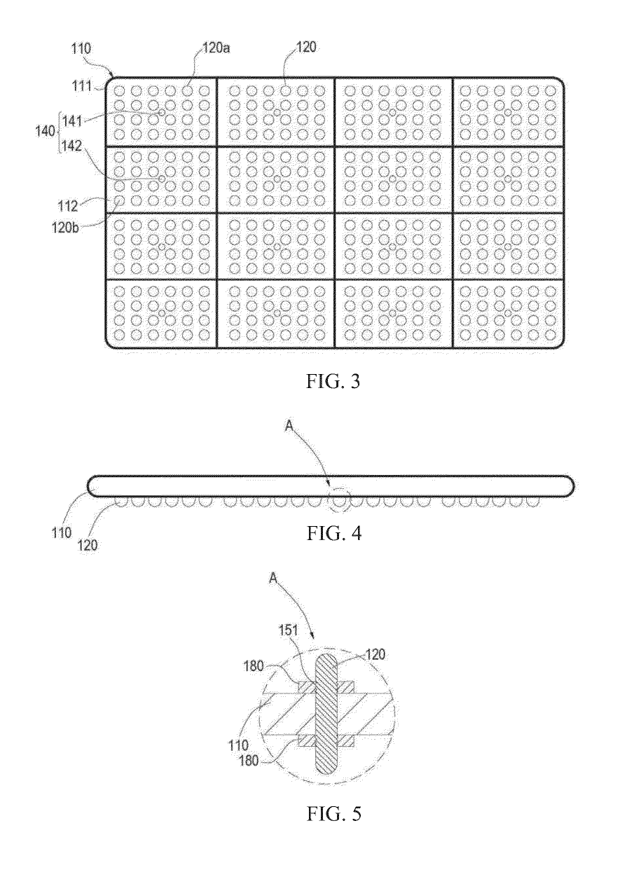

FIG. 1 is a perspective view of a radio frequency (RF) heating device according to a first embodiment of the present invention. FIG. 2 is a structural block diagram of an RF heating device according to a first embodiment of the present invention. FIG. 3 is a top view showing electrodes arrangement according to one embodiment of the present invention. FIG. 4 is a side view of electrodes and a flexible pad of FIG. 3. The RF heating device 100 includes a flexible pad 110, a plurality of electrodes 120, and an RF power generator unit 130. The electrodes 120 are arranged in a form of array and disposed on one side of the flexible pad 110 so as to emit RF energy. The RF energy is electromagnetic wave energy and usually has a frequency between 0.1 and 100 MHz. Preferably, the frequency of RF energy is between 0.1 and 10 MHz. The RF power generator unit 130 is electrically connected to the electrodes 120 for providing the electrodes 120 with RF energy. Preferably, the electrodes 120 are disposed on one side of the flexible pad 110 in a same density, so as to emit the RF energy uniformly.

FIG. 5 is a cross-section view of an enlarged portion A of FIG. 4. The surface of each electrode 120 is provided with a thread 151 and at least one nut 180 used for engaging with the thread 151, so that the electrodes 120 can be fixed in the side of the flexible pad 110. In the embodiment, the electrodes 120 pass through the flexible pad 110, and can be fixed on the flexible pad 110 by using a nut 180 respectively engaging with the top and the bottom ends of the electrode 120.

Please refer to FIG. 2 again, the RF heating device 100 further includes a sensing unit 140, a control unit 150, and a human-machine interface 160. The sensing unit 140 is disposed on the one side of the flexible pad 110 so as to sense an external signal, for example, the sensing unit 140 may be a temperature sensing unit 141 (please refer to FIG. 3) to sense a temperature which can be used as an external signal. In another embodiment, the sensing unit 140 may be a power sensing unit 142 (please refer to FIG. 3) for receiving parts of the RF energy, so as to sense the amount of power loss of the RF energy which can be used as the external signal. The amount of power loss of the RF energy indicates that when the electrodes 120 emit RF energy to skin, one part of the RF energy will be absorbed by the skin and the other part of the RF energy will be lost to air which causes waste of energy. The power sensing unit 142 is mainly used to sense the RF energy lost to the air. The control unit 150 is electrically connected to the RF power generator unit 130 and the sensing unit 140. The control unit 150 is used to control the amount of the RF energy provided by the RF power generator unit 130 to the electrodes 120 according to the external signal. The human-machine interface 160 is electrically connected to the control unit 150, and is used to send a control signal to the control unit 150. The control unit 150 controls the amount of the RF energy provided by the RF power generator unit 130 to the electrodes 120 according to the control signal. In addition, the RF power generator unit 130, the control unit 150, and the human-machine interface 160 can be combined to a processor 170. Preferably, the processor 170 has a display function.

Please refer to FIG. 3 again, in the embodiment, the flexible pad 110 can be divided into a plurality of areas, and includes a plurality of different RF energies. However, for convenience of explanation, in this embodiment, the flexible pad 110 only includes a first area 111 and a second area 112, and the different RF energies only include a first RF energy and a second RF energy, but are not limited thereto. The first RF energy is emitted by the electrodes 120a on the first area 111, the second RF energy is emitted by the electrodes 120b on the second area 112, and the amounts of the first RF energy and the second RF energy are different. The first RF energy and the second RF energy may be controlled by a human-machine interface, so that different amount of the RF energy can be generated according to skin thicknesses of different skin portions, or according to a user's need. For instance, when the amount of the first RF energy is larger than that of the second RF energy, the first RF energy can be used corresponding to the areas with thicker skin, and the second RF energy can be used corresponding to the areas with thinner skin.

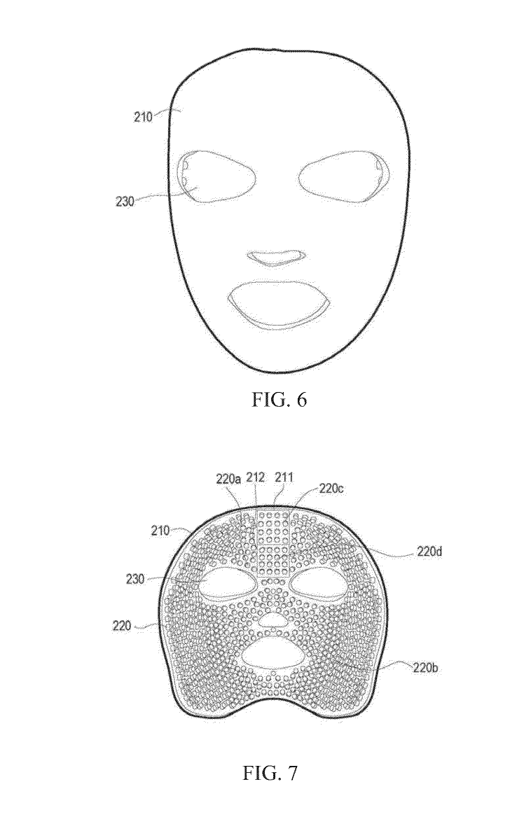

Please refer to FIGS. 6 and 7, they illustrate an RF heating device according to a second embodiment of the present invention. The difference between the RF heating devices of the second embodiment and the first embodiment is that: the electrodes arrangement of the RF heating device in the second embodiment is different from that of the RF heating device in the first embodiment. In addition, the flexible pad is a mask-type flexible pad 210 and has four openings 230 which correspond to the positions of eyes, nose, and mouth. The electrodes 220 of the mask-type flexible pad 210 can be disposed on one side of the mask-type flexible pad 210 in different density. For instance, the electrodes 220 can be disposed on the one side of the mask-type flexible pad 210 in a first density and a second density, and the first density is higher than the second density. The electrodes 220b arranged in the first density can be disposed on the positions corresponding to the skin areas with thicker skin, and the electrodes 220a arranged in the second density can be disposed on the positions corresponding to the skin areas with thinner skin. For example, the skin is thicker in the cheek area so that the electrodes 220b are disposed in the first density; however, the skin is thinner in the eye areas so that the electrodes 220a are disposed in the second density.

In another embodiment, the mask-type flexible pad may be divided into a plurality of areas, and includes a plurality of different RF energies. For convenient explanation, the mask-type flexible pad only includes a first area 211 and a second area 212, and the RF energies only include a first RF energy and a second RF energy, but are not limited thereto. The first RF energy is emitted by the electrodes 220c on the first area, the second RF energy is emitted by the electrodes 220d on the second area, and the first RF energy and the second RF energy are different. For example, the second area 212 is located around the eyes, and the skin around the eyes is thinner, so that the second RF energy is small; however, the skin around the forehead is thicker, and the first area is located around the forehead, so that the first RF energy is large. In addition, the fixation of electrodes, the control element for controlling RF energy and the method therefor are identical to those shown in FIGS. 1 and 3, so that they are not repeatedly described herein.

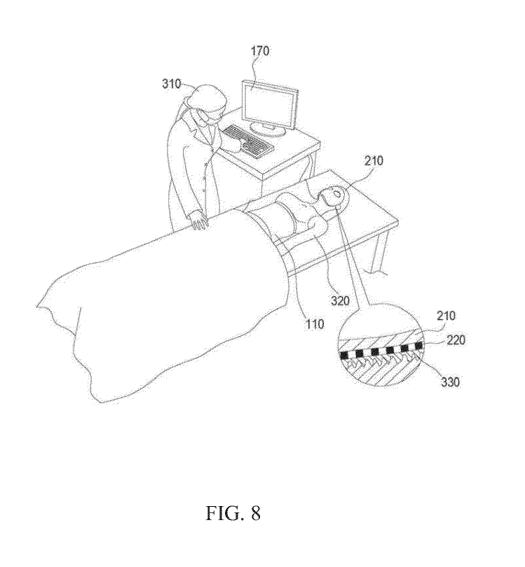

FIG. 8 is a schematic view showing an RF heating device in use according to the first and the second embodiments of the present invention. Please also refer to FIGS. 2, 3 and 7. Before a doctor 310 performs a treatment, the flexible pad 110 and the electrodes 120 of the first embodiment is covered on the abdominal region of a patient 320, and the mask-type flexible pad 210 and the electrodes 220 of the second embodiment is covered on the face of the patient 320. The electrodes 120 on the flexible pad 110 and the electrodes 220 on the mask-type flexible pad 210 can be easily attached to the skin 330 according to the shape of the skin 330. When the flexible pad 110 and the mask-type flexible pad 210 are properly covered, the doctor 310 inputs parameters through the human-machine interface 160 of the processor 170, and the human-machine interface 160 sends a control signal to the control unit 150 according to the parameters. The control unit 150 controls the RF power generator unit 130 according to the control signal, such that the RF power generator unit 130 provides electrical power to the electrodes 120 on the flexible pad 110 and the electrodes 220 on the mask-type flexible pad 210. The parameters may be RF energy parameters for different areas in the flexible pad 110 and the mask-type flexible pad 210 (for example, the first RF energy of the first area and the second RF energy of the second area), time parameters of power supply for electrodes or the temperature parameters of skin.

Please refer to FIGS. 2 and 3, during the treatment, the temperature sensing unit 141 senses the temperature of each portion in the surface of skin 330. Then, the temperature data will be feedback to the control unit 150 by an external signal. If the temperature is too high or too low in some portions, the control unit 150 can reduce or increase the amount of the

RF energy in these portions so as to control the surface of the skin 330 to approach a preset temperature or less than the preset temperature. The power sensing unit 142 senses the amount of power loss of the RF energy from each portion. The data of power loss of the RF energy may be feedback to the control unit 150 by an external signal. If the amount of power loss is too high or too low in some portions, the control unit can reduce or increase the amount of the RF energy in these portions so as to control the power loss within a predetermined tolerance range.

The RF heating device of the present invention can be used in an automatic medical cosmetics treatment for large skin area, and is different from conventional RF heating products. The RF heating device of the present invention can guide RF energy to skin tissues by using a plurality of electrodes disposed on a flexible pad, so as to raise the temperatures of skin tissues to promote the contracts of dermal collagen, elastic fibers and other tissues due to heat absorption. In addition, the area of the flexible pad is adjustable and can be cut into a shape corresponding to each part of human body. For example, the flexible pad can be cut into a shape similar to face. Moreover, when the RF heating device of the present invention is used in a treatment for a large skin area, for example, abdominal region, it can reduce the treating time.

Although the present invention is disclosed as the preferred embodiment described above, however, it is not used to limit the present invention. The equivalent alternatives of the alterations and modifications made by any skilled in the art without departing from the spirit and scope of the present invention still fall in the scope of the claims of the present invention.

What is claimed is:

1. A radio frequency (RF) heating device, comprising:
a flexible pad;
a plurality of electrodes, disposed on one side of the flexible pad for emitting an RF energy; and
an RF generator unit, electrically connected to the electrodes for providing the electrodes with the RF energy.

2. The RF heating device according to claim 1, wherein the flexible pad is a mask-type flexible pad, and the electrodes are disposed on the side of the mask-type flexible pad in different density.

3. The RF heating device according to claim 2, wherein the electrodes are disposed on the side of the flexible pad in a first density and a second density, the first density is higher than the second density, the electrodes arranged in the first density is disposed on positions corresponding to thicker skin areas, and the electrodes arranged in the second density are disposed on positions corresponding to thinner skin areas.

4. The RF heating device according to claim 1, further comprises:
a sensing unit, disposed on the side of the flexible pad and used for sensing an external signal; and
a control unit, electrically connected to the RF power generator unit and the sensing unit, and used for controlling an amount of the RF energy provided by the RF power generator unit to the electrodes according to the external signal.

5. The RF heating device according to claim 2, further comprises:
a sensing unit, disposed on the side of the flexible pad and used for sensing an external signal; and
a control unit, electrically connected to the RF power generator unit and the sensing unit, and used for controlling an amount of the RF energy provided by the RF power generator unit to the electrodes according to the external signal.

6. The RF heating device according to claim 4, wherein the sensing unit is a temperature sensing unit for sensing a temperature, and the temperature is used as the external signal.

7. The RF heating device according to claim 4, wherein the sensing unit is a power sensing unit for receiving parts of the RF energy to sense an amount of power loss of the RF energy, and the amount of power loss of the RF energy is used as the external signal.

8. The RF heating device according to claim 4, further comprises a human-machine interface electrically connected to the control unit, and used to send a control signal to the control unit, and the control unit controls an amount of the RF energy provided by the RF power generator unit to the electrodes according to the control signal.

9. The RF heating device according to claim 1, wherein the flexible pad comprises a first area and a second area, the RF energy comprises a first RF energy and a second RF energy, the first RF energy is emitted by the electrodes on the first area, the second RF energy is emitted by the electrodes on the second area, and the first RF energy and the second energy are different.

10. The RF heating device according to claim 9, wherein the first RF energy is larger than t the second RF energy, the first RF energy is used corresponding to areas with thicker skin and the second RF energy is used corresponding to areas with thinner skin.

11. The RF heating device according to claim 2, wherein the flexible pad comprises a first area and a second area, the RF energy comprises a first RF energy and a second RF energy, the first RF energy is emitted by the electrodes on the first area, the second RF energy is emitted by the electrodes on the second area, and the first RF energy and the second energy are different.

12. The RF heating device according to claim 11, wherein the first RF energy is larger than t the second RF energy, the first RF energy is used corresponding to areas with thicker skin and the second RF energy is used corresponding to areas with thinner skin.

13. The RF heating device according to claim 1, wherein a surface of the electrode is provided with a thread and at least one nut used for engaging with the thread, so that the electrodes are fixed in the side of the flexible pad.

14. The RF heating device according to claim 2, wherein a surface of the electrode is provided with a thread and at least one nut used for engaging with the thread, so that the electrodes are fixed in the side of the flexible pad.
